(19) United States
(12) Patent Application Publication
(10) Pub. No.: US 2004/0009483 A1
(43) Pub. Date: Jan. 15, 2004

(54) Method of linear mRNA amplification using total RNA

(76) Inventors: Diane D. Ilsley, San Jose, CA (US); Homaira Haidari, Fremont, CA (US)

Correspondence Address: AGILENT TECHNOLOGIES, INC., INTELLECTUAL PROPERTY ADMINISTRATION, LEGAL DEPT., P.O. BOX 7599, M/S DL429, LOVELAND, CO, 80537-0599, US

(21) Appl. No.: 10/195,035

(22) Filed: Jul. 12, 2002

(51) Int. Cl. C12Q 1/68; C12P 19/34

(52) U.S. Cl. 435/6; 435/91.2

(57) ABSTRACT

Methods for linearly amplifying mRNA using total RNA to produce antisense RNA are provided. In the subject methods, mRNA is converted to double-stranded cDNA using a promoter-primer having a poly-dT primer site linked to a promoter sequence so that the resulting double-stranded cDNA is recognized by an RNA polymerase. A feature of the subject methods is that the source of mRNA employed in this conversion step is total RNA and the conversion step lasts at least about 4 hours. The resultant double-stranded cDNA is then transcribed into antisense RNA. The subject methods find use a variety of different applications in which the preparation of linearly amplified amounts of antisense RNA is desired. Also provided are kits for practicing the subject methods.

### TECHNICAL FIELD The technical field of this invention is the enzymatic amplification of nucleic acids.

### BACKGROUND OF THE INVENTION

The characterization of cellular gene expression finds application in a variety of disciplines, such as in the analysis of differential expression between different tissue types, different stages of cellular growth or between normal and diseased states. Fundamental to differential expression analysis is the detection of different mRNA species in a test population, and the quantitative determination of different mRNA levels in that test population. However, the detection of rare mRNA species is often complicated by one or more of the following factors: cell heterogeneity, paucity of material, or the limits of detection of the assay method. Thus, methods which amplify heterogeneous populations of mRNA that do not introduce significant changes in the relative amounts of different mRNA species facilitate this technology.

A number of methods for the amplification of nucleic acids have been described. Such methods include the “polymerase chain reaction” (PCR) (Mullis et al., U.S. Pat. No. 4,683,195), and a number of transcription-based amplification methods (Malek et al., U.S. Pat. No. 5,130,238; Kacian and Fultz, U.S. Pat. No. 5,399,491; Burg et al., U.S. Pat. No. 5,437,990). Each of these methods uses primer-dependent nucleic acid synthesis to generate a DNA or RNA product, which serves as a template for subsequent rounds of primer-dependent nucleic acid synthesis. Each process uses (at least) two primer sequences complementary to different strands of a desired nucleic acid sequence and results in an exponential increase in the number of copies of the target sequence. These amplification methods can provide enormous amplification (up to billion-fold). However, these methods have limitations that make them not amenable for gene expression monitoring applications. First, each process results in the specific amplification of only the sequences that are bounded by the primer binding sites. Second, exponential amplification can introduce significant changes in the relative amounts of specific target species—small differences in the yields of specific products (for example, due to differences in primer binding efficiencies or enzyme processivity) become amplified with every subsequent round of synthesis.

Amplification methods that utilize a single primer are amenable to the amplification of heterogeneous mRNA populations. The vast majority of mRNAs carry a homopolymer of 20-250 adenosine residues on their 3′ ends (the poly-A tail), and the use of poly-dt primers for cDNA synthesis is a fundamental tool of molecular biology. “Single-primer amplification” protocols have been reported (see e.g. Kacian et al., U.S. Pat. No. 5,554,516; Van Gelder et al., U.S. Pat. No. 5,716,785). The methods reported in these patents utilize a single primer containing an RNA polymerase promoter sequence and a sequence complementary to the 3′-end of the desired nucleic acid target sequence(s) (“promoter-primer”). In both methods, the promoter-primer is added under conditions where it hybridizes to the target sequence(s) and is converted to a substrate for RNA polymerase. In both methods, the substrate intermediate is recognized by RNA polymerase, which produces multiple copies of RNA complementary to the target sequence(s) (“antisense RNA”). Each method uses, or could be adapted to use, a primer containing poly-dt for amplification of heterogeneous mRNA populations.

Amplification methods that proceed linearly during the course of the amplification reaction are less likely to introduce bias in the relative levels of different mRNAs than those that proceed exponentially. In the method described in U.S. Pat. No. 5,554,516, the amplification reaction contains a nucleic acid target sequence, a promoter-primer, an RNA polymerase, a reverse transcriptase, and reagent and buffer conditions sufficient to allow amplification. The amplification proceeds in a single tube under conditions of constant temperature and ionic strength. Under these conditions, the antisense RNA products of the reaction can serve as substrates for further amplification by non-specific priming and extension by the RNA-dependent DNA polymerase activity of reverse transcriptase. As such, the amplification described in U.S. Pat. No. 5,554,516 proceeds exponentially. In contrast, in specific examples described in U.S. Pat. No. 5,716,785, cDNA synthesis and transcription occur in separation reactions separated by phenol/chloroform extraction and ethanol precipitation (or dialysis), which may incidentally allow for the amplification to proceed linearly since the RNA products cannot serve as substrates for further amplification.

The method described in U.S. Pat. No. 5,716,785 has been used to amplify cellular mRNA for gene expression monitoring (for example, R.N. Van Gelder et al. (1990), Proc. Natl. Acad. Sci. USA 87, 1663; D. J. Lockhart et al. (1996), Nature Biotechnol. 14,1675). However, this procedure is not readily 10 amenable to high throughput processing. In preferred embodiments of the method described in U.S. Pat. No. 5,716,785, poly-A mRNA is primed with a promoter-primer containing poly-dt and converted into double-stranded cDNA using a method described by Gubler and Hoffman (U. Gubler and B. J. Hoffman (1983), Gene 25, 263-269) and popularized by commercially available kits for cDNA synthesis. Using this method for cDNA synthesis, first strand synthesis is performed using reverse transcriptase and second strand. cDNA is synthesized using RNaseH and DNA polymerase I. After phenol/chloroform extraction and dialysis, double-stranded cDNA is transcribed by RNA polymerase to yield antisense RNA product.

One problem with the above-described methods is that purified mRNA is used as the input RNA. Isolation of mRNA directly from cells and total RNA can result in significant losses of low abundant messages and a portion of the overall population. In addition, the process is time consuming and the yields are low, especially from a low number of cells. Polyadenylated RNA can be isolated directly from lysed cells, but guanidine salts used to inhibit RNAses are highly disruptive of binding and can interfere with mRNA binding to an oligodt support.

Accordingly, there is interest in the development of improved methods of antisense RNA amplification. Of particular interest would be the development of a protocol which allowed for the use of total RNA as opposed to isolated mRNA as the input RNA.

Relevant Literature U.S. patents disclosing methods of antisense RNA synthesis include: U.S. Pat. Nos. 6,132,997; 5,932,451; 5,716,785; 5,554,516; 5,545,522; 5,437,990; 5,130,238; and 5,514,545. Antisense RNA synthesis is also discussed in Phillips and Eberwine (1996), Methods: A companion to Methods in Enzymol. 10, 283; Eberwine et al. (1992), Proc., Natl., Acad. Sci. USA 89, 3010; Eberwine (1996), Biotechniques 20, 584; and Eberwine et al. (1992), Methods in Enzymol. 216, 80.

### SUMMARY OF THE INVENTION

Methods for linearly amplifying mRNA using total RNA to produce antisense RNA are provided. In the subject methods, mRNA is converted to double-stranded cDNA using a promoter-primer having a poly-dT primer site linked to a promoter sequence so that the resulting double-stranded cDNA is recognized by an RNA polymerase. A feature of the subject methods is that the source of mRNA employed in this conversion step is total RNA and the conversion step lasts at least about 4 hours. The resultant double-stranded cDNA is then transcribed into antisense RNA. The subject methods find use a variety of different applications in which the preparation of linearly amplified amounts of antisense RNA is desired. Also provided are kits for practicing the subject methods.

### DESCRIPTION OF THE SPECIFIC EMBODIMENTS

Methods for linearly amplifying mRNA using total RNA to produce antisense RNA are provided. In the subject methods, mRNA is converted to double-stranded cDNA using a promoter-primer having a poly-dT primer site linked to a promoter sequence so that the resulting double-stranded cDNA is recognized by an RNA polymerase. A feature of the subject methods is that the source of mRNA employed in this conversion step is total RNA and the conversion step lasts at least about 4 hours. The resultant double-stranded cDNA is then transcribed into antisense RNA. The subject methods find use a variety of different applications in which the preparation of linearly amplified amounts of antisense RNA is desired. Also provided are kits for practicing the subject methods.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the invention components that are described in the publications which might be used in connection with the presently described invention.

As summarized above, the present invention provides methods of preparing amplified amounts of antisense RNA from mRNA using total RNA as the mRNA source, as well as kits for use in practicing the subject methods. In further describing the present invention, the subject methods are discussed first in greater detail, followed by a review of representative kits for use in practicing the subject methods.

Methods

The subject invention provides methods for linearly amplifying mRNA into antisense RNA. As such, the subject invention provides methods of producing amplified amounts of antisense RNA from an initial amount of mRNA. By amplified amounts is meant that for each initial mRNA, multiple corresponding antisense RNAs are produced, where the term antisense RNA is defined here as ribonucleic acid complementary to the initial mRNA. By corresponding is meant that the antisense RNA shares a substantial amount of sequence identity with the sequence complementary to the mRNA (i.e. the complement of the initial mRNA), where substantial amount means at least 95% usually at least 98% and more usually at least 99%, where sequence identity is determined using the BLAST algorithm, as described in Altschul et al. (1990), J. Mol. Biol. 215:403410 (using the published default setting, i.e. parameters w=4, t=17). Generally, the number of corresponding antisense RNA molecules produced for each initial mRNA during the subject linear amplification methods will be at least about 10, usually at least about 50 and more usually at least about 100, where the number may be as great as 600 or greater, but often does not exceed about 1000.

In the first step of the subject methods, an initial mRNA sample is subjected to a series of enzymatic reactions under conditions sufficient to ultimately produce double-stranded DNA for each initial mRNA in the sample that is amplified. During this first step, an RNA polymerase promoter region is incorporated into the resultant product, which region is employed in the second step of the subject methods, i.e. the transcription step described in greater detail infra.

A feature of the subject methods is that the total RNA sample, i.e., a total RNA preparation, is employed as the source of mRNA, where the total RNA sample will typically be derived from a physiological source. The physiological source may be derived from a variety of eukaryotic sources, with physiological sources of interest including sources derived from single-celled organisms such as yeast and multicellular organisms, including plants and animals, particularly mammals, where the physiological sources from multicellular organisms may be derived from particular organs or tissues of the multicellular organism, or from isolated cells derived therefrom. In obtaining the total RNA preparation from the physiological source from which it is derived, any convenient protocol for isolation of total RNA from the initial physiological source may be employed. Methods of isolating RNA from cells, tissues, organs or whole organisms are known to those of skill in the art and include those described in Maniatis et al. (1989), Molecular Cloning: A Laboratory Manual 2d Ed. (Cold Spring Harbor Press).

Depending on the nature of the primer employed during first strand synthesis, as described in greater detail below, the subject methods can be used to produce amplified amounts of antisense RNA corresponding to substantially all of the mRNA present in the initial sample, or to a proportion or fraction of the total number of distinct mRNAs present in the initial sample. By substantially all of the mRNA present in the sample is meant more than 90%, usually more than 95%, where that portion not amplified is solely the result of inefficiencies of the reaction or the enzyme and not intentionally excluded from amplification.

The promoter-primer employed in the amplification reaction includes: (a) a poly-dt region for hybridization to the poly-A tail of the mRNA; and (b) an RNA polymerase promoter region 5′ of the poly-dT region that is in an orientation capable of directing transcription of antisense RNA. In certain embodiments, the primer will be a “lock-dock” primer, in which immediately 3′ of the poly-dt region is either a “G”, “C”, or “A” such that the primer has the configuration of 3′-XTTTTTTT . . . . . . 5′, where X is “G”, “C”, or “A”. The poly-dT region is sufficiently long to provide for efficient hybridization to the poly-A tail, where the region typically ranges in length from 10-50 nucleotides in length, usually 10-25 nucleotides in length, and more usually from 14 to 20 nucleotides in length.

A number of RNA polymerase promoters may be used for the promoter region of the first strand cDNA primer, i.e. the promoter-primer. Suitable promoter regions will be capable of initiating transcription from an operationally linked DNA sequence in the presence of ribonucleotides and an RNA polymerase under suitable conditions. The promoter will be linked in an orientation to permit transcription of antisense RNA. A linker oligonucleotide between the promoter and the DNA may be present, and if, present, will typically comprise between about 5 and 20 bases, but may be smaller or larger as desired. The promoter region will usually comprise between about 15 and 250 nucleotides, preferably between about 17 and 60 nucleotides, from a naturally occurring RNA polymerase promoter or a consensus promoter region, as described in Alberts et al. (1989) in Molecular Biology of the Cell, 2d Ed. (Garland Publishing, Inc.). In general, prokaryotic promoters are preferred over eukaryotic promoters, and phage or virus promoters most preferred. As used herein, the term “operably linked” refers to a functional linkage between the affecting sequence (typically a promoter) and the controlled sequence (the mRNA binding site). The promoter regions that find use are regions where RNA polymerase binds tightly to the DNA and contain the start site and signal for RNA synthesis to begin. A wide variety of promoters are known and many are very well characterized. Representation promoter regions of particular interest include T7, T3 and SP6 as described in Chamberlin and Ryan, The Enzymes (ed. P. Boyer, Academic Press, New York) (1982) pp 87-108.

Where one wishes to amplify only a portion of the mRNA species in the sample, one may optionally provide for a short arbitrary sequence 3′ of the poly-dT region, where the short arbitrary sequence will generally be less than 5 nucleotides in length and usually less than 2 nucleotides in length, where the dNTP immediately adjacent to the poly-dT region will not be a T residue and usually the sequence will comprise no T residue. Such short 3′ arbitrary sequences are described in Ling and Pardee (1992), Science 257, 967.

The promoter-primer described above and throughout this specification may be prepared using any suitable method, such as, for example, the known phosphotriester and phosphite triester methods, or automated embodiments thereof. In one such automated embodiment, dialkyl phosphoramidites are used as starting materials and may be synthesized as described by Beaucage et al. (1981), Tetrahedron Letters 22, 1859. One method for synthesizing oligonucleotides on a modified solid support is described in U.S. Pat. No. 4,458,066. It is also possible to use a primer that has been isolated from a biological source (such as a restriction endonuclease digest). The primers herein are selected to be “substantially” complementary to each specific sequence to be amplified, i.e.; the primers should be sufficiently complementary to hybridize to their respective targets. Therefore, the primer sequence need not reflect the exact sequence of the target, and can, in fact be “degenerate.” Non-complementary bases or longer sequences can be interspersed into the primer, provided that the primer sequence has sufficient complementarity with the sequence of the target to be amplified to permit hybridization and extension.

In the first step of the subject methods, the oligonucleotide promoter-primer is hybridized with a sufficient amount of an initial total RNA (containing the mRNA to be-amplified) sample (as described above) and the primer-mRNA hybrid is converted to a double-stranded cDNA product that is recognized by an RNA polymerase. The amount of initial total RNA sample that is employed may vary, and may be as low as 6 μg or lower, e.g., about 1 μg or lower, about 500 ng or lower, about 100 ng or lower, etc., where the amount in many embodiments ranges from about 10 ng to about 10 μg, usually from about 500 ng to about 6 μg, and the amount of total RNA sample employed in certain embodiments does not exceed about 20 μg, and often does not exceed about 10 μg.

The promoter-primer is contacted with the mRNA of the total RNA sample under conditions that allow the poly-dT site to hybridize to the poly-A tail present on most mRNA species in the total RNA sample. The resultant duplexes are then maintained under conditions sufficient to produce double-stranded cDNA from the duplexes. As such, the resultant duplexes are maintained in the presence of reagents necessary to, and for a period of time sufficient to, convert the primer-mRNA hybrids to double stranded cDNAs.

The catalytic activities required to convert primer-mRNA hybrid to double-stranded cDNA are an RNA-dependent DNA polymerase activity, a RNaseH activity, and a DNA-dependent DNA polymerase activity. Most reverse transcriptases, including those derived from Moloney murine leukemia virus (MMLV-RT), avian myeloblastosis virus (AMV-RT), bovine leukemia virus (BLV-RT), Rous sarcoma virus (RSV) and human immunodeficiency virus (HIV-RT) catalyze each of these activities. These reverse transcriptases are sufficient to convert primer-mRNA hybrid to double-stranded DNA in the presence of additional reagents which include, but are not limited to: dNTPs; monovalent and divalent cations, e.g. KCl, MgCl₂; sulfhydryl reagents, e.g. dithiothreitol; and buffering agents, e.g. Tris-Cl. Alternatively, a variety of proteins that catalyze one or two of these activities can be added to the cDNA synthesis reaction. For example, MMLV reverse transcriptase lacking RNaseH activity (described in U.S. Pat. No. 5,405,776) which catalyzes RNA-dependent DNA polymerase activity and DNA-dependent DNA polymerase activity, can be added with a source of RNaseH activity, such as the RNaseH purified from cellular sources, including Escherichia coli. These proteins may be added together during a single reaction step, or added sequentially during two or more substeps. Finally, additional proteins that may enhance the yield of double-stranded DNA products may also be added to the cDNA synthesis reaction. These proteins include a variety of DNA polymerases (such as those derived from E. coli, thermophilic bacteria, archaebacteria, phage, yeasts, Neurosporas, Drosophilas, primates and rodents), and DNA Ligases (such as those derived from phage or cellular sources, including T4 DNA Ligase and E. coli DNA Ligase).

In certain embodiments, it is desirable to include one or more detergents, where the detergent enhances the amount of aRNA that is ultimately produced. If a detergent is employed a number of detergent types are useful. Detergents such as bile salts, cholate, deoxycholate, lithocholate may be used. Also useful are ionic detergents such as anionic, cationic or zwitterionic detergents. Guanidine salts, such as Guanidine hydrochloride and Guanidine thiocyanate may be used. Additionally, nonionic surfactants such as polyoxyethylene sorbitol ester or polyoxyethylene p-t octylphenol may be used. Specifically the Tween series, including Tween 20 and Tween 80, and Triton series, including Triton N-101 and NP-40, are preferred. Tween-80 or Triton X-100 are most preferred. While the concentration employed may vary, in many embodiments the concentration ranges from about 0.001% to about 0.1%, and often from about 0.005% to about 0.025%.

Conversion of primer-mRNA hybrid to double-stranded cDNA by reverse transcriptase proceeds through an RNA:DNA intermediate which is formed by extension of the hybridized promoter-primer by the RNA-dependent DNA polymerase activity of reverse transcriptase. The RNaseH activity of the reverse transcriptase then hydrolyzes at least a portion of the RNA:DNA hybrid, leaving behind RNA fragments that can serve as primers for second strand synthesis (Meyers et al., Proc. Nat'l Acad. Sci. USA (1980) 77:1316 and Olsen &Watson, Biochem. Biophys. Res. Commun. (1980) 97:1376). Extension of these primers by the DNA-dependent DNA polymerase activity of reverse transcriptase results in the synthesis of double-stranded cDNA. Other mechanisms for priming of second strand synthesis may also occur, including “self-priming” by a hairpin loop formed at the 3′ terminus of-the first strand cDNA (Efstratiadis et al. (1976), Cell 7, 279; Higuchi et al. (1976), Proc. Natl, Acad, Sci USA 73, 3146; Maniatis et al. (1976), Cell 8, 163; and Rougeon and Mach (1976), Proc. Natl. Acad. Sci. USA 73, 3418; and “non-specific priming” by other DNA molecules in the reaction, i.e. the promoter-primer.

The second strand cDNA synthesis results in the creation of a double-stranded promoter region. The second strand cDNA includes not only a sequence of nucleotide residues that comprise a DNA copy of the mRNA template, but also additional sequences at its 3′ end which are complementary to the promoter-primer used to prime first strand cDNA synthesis. The double-stranded promoter region serves as a recognition site and transcription initiation site for RNA polymerase, which uses the second strand cDNA as a template for multiple rounds of RNA synthesis during the next stage of the subject methods.

Depending on the particular protocol, the same or different DNA polymerases may be employed during the cDNA synthesis step. For example, a single reverse transcriptase, most preferably MMLV-RT, may be used as a source of all the requisite activities necessary to convert primer-mRNA hybrid to double-stranded cDNA. Alternatively, the polymerase employed in first strand cDNA synthesis may be different from that which is employed in second strand cDNA synthesis. Specifically, a reverse transcriptase lacking RNaseH activity (e.g. Superscript II™) may be combined with the primer-mRNA hybrid during a first substep for first strand synthesis. A source of RNaseH activity, such as E. coli RNaseH or MMLV-RT, may be added during a second substep to initiate second strand synthesis. In yet other embodiments, the requisite activities are provided by a plurality of distinct enzymes. The manner is which double-stranded cDNA is produced from the initial mRNA is not critical to certain embodiments of the invention. However, in certain embodiments one employs MMLV-RT, or a combination of Superscript I™ and MMLV-RT, or a combination of Superscript II™ and E. coli RNaseH, for cDNA synthesis as these embodiments yield certain desired results.

A feature of the subject invention is that the above described cDNA synthesis step includes an incubation or reaction period that is sufficiently long to ultimately generate adequate amounts of antisense RNA from the initial total RNA sample. Typically, the incubation or reaction period lasts for a period of time that is at least about 4 hours long, where the incubation or reaction period typically ranges from about 4 to 24 hours, usually from about 4 to 16 hours and more usually from about 4 to 8 hours.

The next step of the subject method is the preparation of antisense RNA from the double-stranded cDNA prepared in the first step. During this step, the double-stranded cDNA produced in the first step is transcribed by RNA polymerase to yield antisense RNA, which is complementary to the initial mRNA target from which it is amplified.

Depending on the particular protocol employed, the subject methods may or may not include a step in which the double-stranded cDNAs produced as described above are physically separated from the reverse transcriptase employed in the cDNA production step prior to the transcription step. As such, in certain embodiments, the cDNAs produced in the first step of the subject methods are separated from the reverse transcriptase employed in this first step prior to the second transcription step described in greater detail below. In these embodiments, any convenient separation protocol may be employed, including the phenol/chloroform extraction and ethanol precipitation (or dialysis), protocol as described in U.S. Pat. Nos. 5,554,516 and U.S. Pat. No. 5,716,785, the disclosures of which are herein incorporated by reference.

In yet other embodiments, the subject methods do not involve a step in which the double-stranded cDNA is physically separated from the reverse transcriptase following double-stranded cDNA preparation. In these embodiments, the reverse transcriptase that is present during the transcription step is rendered inactive. Thus, the transcription step is carried out in the presence of a reverse transcriptase that is unable to catalyze RNA-dependent DNA polymerase activity, at least for the duration of the transcription step. As a result, the antisense RNA products of the transcription reaction cannot serve as substrates for additional rounds of amplification, and the amplification process cannot proceed exponentially.

The reverse transcriptase present during the transcription step may be rendered inactive using any convenient protocol, including those described in U.S. Pat. No. 6,132,997; the disclosure of which is herein incorporated by reference. As described in this reference, the transcriptase may be irreversibly or reversibly rendered inactive. Where the transcriptase is reversibly rendered inactive, the transcriptase is physically or-chemically altered so as to no longer able to catalyze RNA-dependent DNA polymerase activity. The transcriptase may be irreversibly inactivated by any convenient means. Thus, the reverse transcriptase may be heat inactivated, in which the reaction mixture is subjected to heating to a temperature sufficient to inactivate the reverse transcriptase prior to commencement of the transcription step. In these embodiments, the temperature of the reaction mixture and therefore the reverse transcriptase present therein is typically raised to 55° C. to 70° C. for 5 to 60 minutes, usually to about 65° C. for 15 to 20 minutes. Alternatively, reverse transcriptase may irreversibly inactivated by introducing a reagent into the reaction mixture that chemically alters the protein so that it no longer has RNA-dependent DNA polymerase activity. In yet other embodiments, the reverse transcriptase is reversibly inactivated. In these embodiments, the transcription may be carried out in the presence of an inhibitor of RNA-dependent DNA polymerase activity. Any convenient reverse transcriptase inhibitor may be employed which is capable of inhibiting RNA-dependent DNA polymerase activity a sufficient amount to provide for linear amplification. However, these inhibitors should not adversely affect RNA polymerase activity. Reverse transcriptase inhibitors of interest include ddNTPs, such as ddATP, ddCTP, ddGTP or ddTTP, or a combination thereof, the total concentration of the inhibitor typically ranges from about 50 μM to 200 μM.

Regardless of whether the cDNA is separated from the reverse transcriptase prior to the transcription step, for the transcription step, the presence of the RNA polymerase promoter region on the double-stranded cDNA is exploited for the production of antisense RNA. To synthesize the antisense RNA, the double-stranded DNA is contacted with the appropriate RNA polymerase in the presence of the four ribonucleotides, under conditions sufficient for RNA transcription to occur, where the particular polymerase employed will be chosen based on the promoter region present in the double-stranded DNA, e.g. T7 RNA polymerase, T3 or SP6 RNA polymerases, E. coli RNA polymerase, and the like. Suitable conditions for RNA transcription using RNA polymerases are known in the art; see e.g. Milligan and Uhlenbeck (1989), Methods in Enzymol. 180, 51.

Utility

The resultant antisense RNA produced by the subject methods finds use in a variety of applications. For example, the resultant antisense RNA can be used in expression profiling analysis on such platforms as DNA microarrays, for construction of “driver” for subtractive hybridization assays, for cDNA library construction, and the like. Especially facilitated by the subject methods are studies of differential gene expression in mammalian cells or cell populations. The cells may be from blood (e.g., white cells, such as T or B cells) or from tissue derived from solid organs, such as brain, spleen, bone, heart, vascular, lung, kidney, liver, pituitary, endocrine glands, lymph node, dispersed primary cells, tumor cells, or the like. The RNA amplification technology can also be applied to improve methods of detecting and isolating nucleic acid sequences that vary in abundance among different populations using the technique known as subtractive hybridization. In such assays, two nucleic acid populations, one sense and the other antisense, are allowed to mix with one another with one population being present in molar excess (“driver”). Under appropriate conditions, the sequences represented in both populations form hybrids, whereas sequences present in only one population remains single-stranded. Thereafter, various well-known techniques are used to separate the unhybridized molecules representing differentially expressed sequences. The amplification technology described herein may be used to construct large amounts of antisense RNA for use as “driver” in such experiments.

Depending on the particular intended use of the subject antisense RNA, the antisense RNA may be labeled. One way of labeling which may find use in the subject invention is isotopic labeling, in which one or more of the nucleotides is labeled with a radioactive label, such as ³²S, ³²p, ³H, or the like. Another means of labeling is fluorescent labeling in which fluorescently tagged nucleotides, e.g. CTP, are incorporated into the antisense RNA product during the transcription step. Fluorescent moieties which may be used to tag nucleotides for producing labeled antisense RNA include: fluorescein, the cyanine dyes, such as Cy3, Cy5, Alexa 555, Bodipy 630/650, and the like. Other labels may also be employed as are known in the art.

As such, the subject methods of nucleic acid generation find use in nucleic acid analyte detection applications, where the subject methods are employed to generate the nucleic acid analyte. Specific analyte detection applications of interest include hybridization assays in which the nucleic acid produced by the subject methods are hybridized to arrays of probe nucleic acids.

An “array”, unless a contrary intention appears, includes any one-, two- or three-dimensional arrangement of addressable regions bearing a particular chemical moiety or moieties (for example, biopolymers such as polynucleotide sequences) associated with that region. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probes” may be the one which is to be evaluated by the other (thus, either one could be an unknown mixture of polynucleotides to be evaluated by binding with the other). An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

In these assays, a sample of target nucleic acids is first prepared according to the methods described above, where preparation may include labeling of the target nucleic acids with a label, e.g. a member of signal producing system. Following sample preparation, the sample is contacted with an array under hybridization conditions, whereby complexes are formed between target nucleic acids that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

In certain embodiments, the subject methods include a step of transmitting data from at least one of the detecting and deriving steps, as described above, to a remote location. By “remote location” is meant a location other than the location at which the array is present and hybridization occur. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc. As such, in using an array made by the method of the present invention, the array will typically be exposed to a sample (for example, a fluorescently labeled analyte, e.g., protein containing sample) and the array then read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER scanner available from Agilent Technologies, Palo Alto, Calif. Other suitable apparatus and methods are described in U.S. patent applications: Ser. No. 09/846,125 “Reading Multi-Featured Arrays” by Dorsel et al.; and Ser. No. 09/430,214 “Interrogating Multi-Featured Arrays” by Dorsel et al. As previously mentioned, these references are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

Kits

Also provided are kits for use in the subject invention, where such kits may comprise containers, each with one or more of the various reagents (typically in concentrated form) utilized in the methods, including, for example, buffers, the appropriate nucleotide triphosphates (e.g. dATP, dCTP, dGTP, dTTP, ATP, CTP, GTP and UTP), reverse transcriptase, RNA polymerase, and the promoter-primer of the present invention. Also present in the kits may be total RNA isolation reagents, e.g., RNA extraction buffer, proteinase digestion buffer; proteinase K, etc. Also present in the kits may be one or more detergents.

Finally, the kits may further include instructions for using the kit components in the subject methods. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc.

The following examples are offered by way of illustration and not by way of limitation.

### EXPERIMENTAL

A. Materials and Methods

1. cDNA synthesis reaction-20 μL

input RNA

1.5 μM T7 Promoter Primer

50 mM Tris-HCL, pH 8.3

3 mM MgCl₂

75 mM KC1

10 mM DTT

500 μM dNTPs (each)

200 ng Random Hexamers

20 U RNaseOUT

200 U MMLV RT

2. Transcription Reaction-80 μL

ds cDNA (20 μL above reaction)

52 mM Tris-HCl, Ph 8.0

15 mM MgCl₂

19 mM KC1

25 mM NaCl

2 mM Spermidine

10 mM DTT

2.5 mM NTP (each)

2000 U T7 RNA polymerase

18 U RNaseOUT

0.12 U Inorganic pyrophosphatase

B. Summary of Results

1. A protocol that employed total RNA in an amplification reaction using conditions described for mRNA was evaluated. It was unexpectedly determined that the yield of a T7 amplification is dependent upon synthesis of the cDNA intermediate, and the rate of this reaction is markedly slower when the input RNA is total RNA compared to polyadenylated RNA. By increasing the time of the cDNA synthesis reaction to a minimum of 4 hours, the total yield of amplified RNA from 6 μg total RNA was found to be similar to the yield obtained from 200 ng polyadenylated RNA (Table 1). Furthermore, the addition of detergent (Triton X-100) also increased the yield of RNA when using total RNA as the input RNA, but had minimal effects on RNA yield when using polyA as the input RNA Using total RNA as the input RNA allows the user to actually use less RNA. It is estimated that the mRNA population is 1-5% of the total RNA, depending upon the cell type. In this example, 6.6 μg total RNA corresponds to 66-330 ng polyA RNA. If the mRNA is only 1%, then using total RNA has actually decreased the mRNA input by 3-fold.

TABLE 1
Yield of RNA 9μg)
Each entry represents an average of 2-6 reactions

| | | | 0.015% |
|---|---|---|---|
| Innut RNA | Yield (μg) | cDNA rxn time (min) | Triton X-100 |
| Hela | | | |
| 200 ng polyA RNA | 57.57 | 120 | − |
| 200 ng polyA RNA | 58.97 | 120 | + |
| 6.6 μg total RNA | 13.66 | 120 | − |
| 6.6 μg total RNA | 17.92 | 120 | + |
| 6.6 μg total RNA | 39.54 | 240 | − |
| 6 6 μg total RNA | 68.33 | 240 | + |
| Spleen | | | |
| 200 ng polyA RNA | 44.58 | 120 | − |
| 6.6 μg total RNA | 39.29 | 240 | + |

As can be seen from the above results, the subject methods generate similar amounts of 15 amplified RNA using around 6 μg total RNA relative to 200 ng polyA RNA. In addition, using low amounts of total RNA (1-6 μg) significantly reduces the amount of mRNA used as the input. Total RNA is faster and easier to purify, resulting in higher yields relative to mRNA purification, and can be isolated from a minimum of 100 cells.

The above results and discussion demonstrate that novel and improved methods of producing linearly amplified amounts of antisense RNA from an initial mRNA source that is total RNA are provided. The subject methods provide for an improvement over prior methods of producing antisense RNA in that the one can use decreased amounts of input RNA and need not employ purified mRNA as the input RNA. As such, the subject methods represent a significant contribution to the art.

All publications and patent application cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method for producing linearly amplified amounts of antisense RNA from total RNA, said method comprising:
(a) producing double-stranded cDNA from total RNA by maintaining said total RNA in the presence of reverse transcriptase reagents under reverse transcriptase conditions for a period of at least about 4 hours, wherein one terminus of said double-stranded cDNA comprises an RNA polymerase promoter region; and
(b) transcribing said double-stranded cDNA into antisense RNA.

2. The method according to claim 1, wherein said double-stranded cDNA is separated from reverse transcriptase prior to said transcribing step (b).

3. The method according to claim 1, wherein said transcribing step (b) occurs in the presence of a reverse transcriptase that is incapable of RNA-dependent DNA polymerase activity during said transcribing step.

4. The method according to claim 1, wherein said reverse transcriptase reagents further include a detergent.

5. The method according to claim 1, wherein the amount of said total RNA employed in said producing step (a) does not exceed about 20 μg.

6. The method according to claim 1, wherein said producing step (a) comprises a single cDNA synthesis step, wherein the same polymerase is employed for the synthesis of first and second cDNA strands.

7. The method according to claim 1, wherein said producing step (a) comprises a first strand cDNA synthesis step and a second strand cDNA synthesis step.

8. The method according to claim 7, wherein a first polymerase is employed for synthesis of said first strand cDNA and a second polymerase is employed for synthesis of said second strand cDNA, wherein said first polymerase is lacking RNaseH activity.

9. The method according to claim 1, wherein said producing step (a) employs a promoter-primer comprising an mRNA binding site linked to a promoter sequence.

10. The method according to claim 1, wherein said producing step (a) comprises:
(i) contacting total RNA with a promoter-primer under conditions wherein said mRNA forms a complex with said promoter-primer, wherein said promoter-primer comprises an mRNA binding site linked to a promoter sequence; and
(ii) converting said complex to double-stranded cDNA using a combination of RNA-dependent DNA polymerase activity, RNaseH activity and DNA-dependent DNA polymerase activity.

11. The method according to claim 10, wherein said RNA-dependent DNA polymerase activity, RNaseH activity and DNA-dependent DNA polymerase activity are contributed by a single polymerase.

12. The method according to claim 11, wherein said polymerase is the reverse transcriptase of Moloney Murine leukemia virus (MMLV-RT).

13. The method according to claim 11, wherein said polymerase is the reverse transcriptase of avian myeloblastosis virus (AMV-RT).

14. The method according to claim 1, wherein said RNA polymerase promoter region is the T7 promoter or the T3 promoter.

15. A kit for use in linearly amplifying mRNA into antisense RNA, said kit comprising:
an oligonucleotide promoter-primer comprising an RNA polymerase promoter sequence; and
instructions for practicing the method according to claim 1.

16. The kit according to claim 15, wherein said kit further comprises at least one polymerase.

17. The kit according to claim 15, wherein said kit further comprises an RNA polymerase.

18. A method of detecting the presence of a nucleic acid analyte in a sample of linearly amplified amounts of antisense RNA produced from total RNA according to claim 1, said method comprising:
(a) contacting said sample suspected with a nucleic acid array;
(b) detecting any binding complexes on the surface of the said array to obtain binding complex data; and
(c) determining the presence of said nucleic acid analyte in said sample using said binding complex data.

19. The method according to claim 18, wherein said method further comprises a data transmission step in which a result from a reading of the array is transmitted from a first location to a second location.

20. A method according to claim 19, wherein said second location is a remote location.

21. A method comprising receiving data representing a result of a reading obtained by the method of claim 18.

22. A hybridization assay comprising the steps of:
(a) contacting at least one labeled target nucleic acid sample of linearly amplified amounts of antisense RNA produced from total RNA according to the method of claim 1 with a nucleic acid array to produce a hybridization pattern; and
(b) detecting said hybridization pattern.

23. The method according to claim 22, wherein said method further comprises washing said array prior to said detecting step.
